(19) United States
(12) Patent Application Publication
(10) Pub. No.: US 2003/0096030 A1
(43) Pub. Date: May 22, 2003

(54) Extracting materials from the shell of Xanthoceras sorbifolia Bunge and applying the extracted materials to making drugs and functional foods

(76) Inventors: Songjiang Wang, Thousand Oaks, CA (US); Baizhen Yang, Shenyang (CN)

Correspondence Address: Songjiang Wang, P.O. Box 1692, Thousand Oaks, CA, 91358, US

(21) Appl. No.: 10/285,209

(22) Filed: Oct. 31, 2002

(30) Foreign Application Priority Data
Oct. 31, 2001 (CN) 01133387.1

(51) Int. Cl. A61K 35/78; A61K 31/70; A61K 31/56

(52) U.S. Cl. 424/776; 514/23; 514/170

(57) ABSTRACT

This invention proposed applications of the extracted materials from shell of Xanthoceras sorbifolia Bunge to making drugs for enhancing brain functions. The extracted materials from the shell of X. sorbifolia include 2˜10 percent of crude fats, 15˜20 percent of crude protein, 15˜25 percent of Bunkankasaponin A. B. C. D., 20˜30 percent of sugar and 7˜12 percent of water as well as two kinds of sterols of (3β,5α,20R,24S)-stigmasta-7, trans-22-dien-3-ol and (3β,5α,20R,24R)-stigmasta-7-en-3-ol. The applications of this invention shows evidently effective for curing diseases of mental retardation and deficiency, dementia, enuresis, and increasing body's ability of resistance to activity of glycosuria. The extracted materials can also be made of functional foods for increasing brain's functions.

### TECHNICAL FIELD

This invention relates to drug and functional food raw materials extracted from the shell of Xanthoceras sorbifolia Bunge.

### BACKGROUND OF INVENTION

Xanthoceras sorbifolia Bunge, also named as Xanthoceras sorbifolium, is a kind of woody oil-bearing plant of Xanthoceras Sapindaceae. It originated primarily at loess plateau in North of China, with characters of drought-enduring, cold-resistant, barren-enduring and saline-alkali-enduring. For these features, it has been planted in most provinces of northern China for windbreak and sand fixation.

X. sorbifolia's kernel tastes pleasant and eatable according of a Chinese ancient book named Provide Disaster Relief of Chinese Materia Medrea. It contains up to 60 percent of high quality, edible oils which riches in 14 kinds of unsaturated fatty acids. X. sorbifolia's kernel can be applied to industry and pharmacy as fined grease, plasticizers, scald plaster, medicine for curing epilepsy etc., and, has been used to cure enuresis.

Scientific personnel of Institute of applied Ecology, Chinese Academy of Sciences, have been involved in the research work of extracting Bunkankasaponin A. B. C. D., crude fats, crude protein, from the kernel of X. sorbifolia. It was verified that Bunkankasaponin A. B. C. D. can improve brain function and make medicine for caring mental retardation and deficiency. A patent application has been filed to State Intellectual Patent Office of P.R.C. (SIPO) in Beijing numbered ZL93111010.6.

There was a report in writing that the shell of X. sorbifolia could be used as chemical raw material for manufacturing activated carbon, furfural, wood sugar, ethyl alcohol etc. However, that report was only a deduction based on the composition of the shell of X. sorbifolia. In fact, no one so far treats the shell of X. sorbifolia as chemical raw material. Farmers only use a few of them as ignition firewood. After researching released foreign and domestic documents, no document proposed that X. sorbifolia's shell is capable of making drugs and functional foods. This invention has developed methods to analyze active materials in the X. sorbifolia's shell, to extract active materials from the shell and has proposed applications to utilize the shell.

### DISCLOSURE OF INVENTION

This invention has discovered the active materials in the shell of X. sorbifolia, the methods of extracting active materials from the shell, and proposed applications to make drugs and functional foods to enhance brain function and to cure brain disease.

The active materials extracted from the shell of X. sorbifolia contain 2˜10 percent of crude fats, 15˜20 percent of crude protein, 15˜25 percent of Bunkankasaponin A. B. C. D., 20˜30 percent of sugar and 7˜12 percent of water.

One way of extracting the active materials from the shell of X. sorbifolia is by using Soxhiet method, with which samples are immersed with n-hexane(CH₃(CH₂)₄CH₃). Then, analyze the extracted fatty acids, after the process of esterification, by using GC-MS system(Gas Chromatographs and Mass Spectrometer system). The result shows that the shell of X. sorbifolia contains 21 kinds of fatty acids. Among them, hexanoic acid(caproic acid), heptanoic acid(oenanthic acid), octanoic acid(caprylic acid), nonanoic acid(pelargonic acid), decanoic acid(capric acid), 10-methyl-undecanoic acid, 12-methyl-tetradecanoic acid and heptadecanoic acid, are eight kinds of fatty acids that are not found in the kernel of X. sorbifolia.

The extracts from the shell of X. sorbifolia also include two kinds of sterols: (3β,5α,20R,24S)-stigmasta-7, trans-22-dien-3-ol and (3β,5α,20R,24R)-stigmasta-7-en-3-ol. These two sterols, however, are not found in the kernel of X. sorbifolia or any other parts of X. sorbifolia plant, according to related documents. The chemical compound of 7-en-3- sterol has property of resistance to activity of glycosuria and tumor.

The procedure to extract the active materials from the shell of the X. sorbifolia is as the follows.

Grind the shell of X. sorbifolia, impregnate it using ethanol(ethyl alcohol), alcohol of methanol(methyl alcohol), or n-propanol(n-acetone), extract the mixture several times at defined interval duration, filter the extracted liquid and remove solvent. After drying and grinding the extracted materials, brown granular powder can be obtained. If refine the brown granular powder, yellow-white granular powder will be obtained.

The key issue of this invention is the discovery of which Bunkankasaponin A. B. C. D., crude fats, crude protein and sterol extracted from the shell of X. sorbifolia have healthcare and medication function.

The purposes of using the extracted materials from the shell of X. sorbifolia are to make drugs and functional foods for enhancing brain function. The extracted materials from the shell of X. sorbifolia are ground into granular powder and packed into capsules, tablets, pellets, or powder, which contain 2˜10 percent of crude fats, 15˜20 percent of crude protein, 15˜25 percent of Bunkankasaponin A. B. C. D., 20˜30 percent of sugar, and 7˜12 percent of water and sterols. It can cure mental retardation and deficiency, dementia, enuresis, etc. While absorbing the extracts, people can get various elements of unsaturated fatty acids, amino acid as well as sterols to improve their immunities.

### BRIEF DESCRIPTION OF DRAWINGS

FIG. 1 is a flow diagram of extracting materials from the shell of X. sorbifolia.

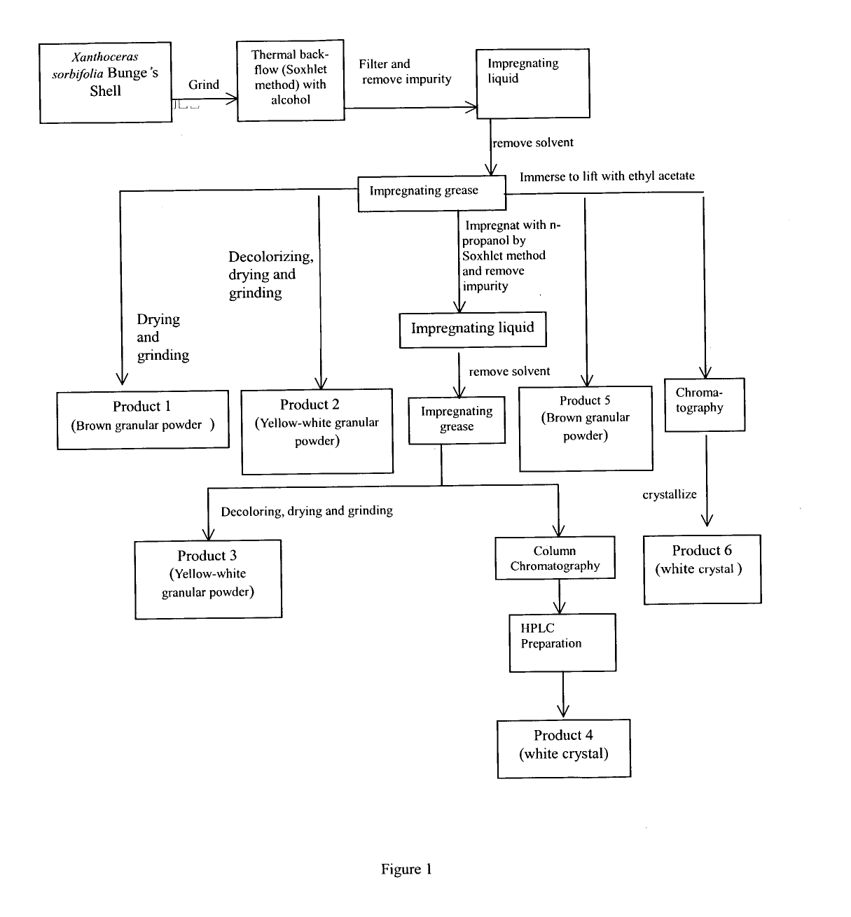

### METHOD OF IMPLEMENTATION

### EXAMPLE 1

Take 2000 g shell of X. sorbifolia, grind it down. Add ethyl alcohol 3 times (in volume) as much as the ground shell. Then extract materials 4 times by using Soxhlet method. Collect impregnation liquid, retrieve solvent. After drying the filtered liquid and grinding the distilled material, 110 g brown granular powder was obtained, which contained (wt %):

| Crude | Crude | | Bunkankasaponin | | | |
|---|---|---|---|---|---|---|
| Fats | protein | Sugar | A. B. C. D. | Water | Sterols | Others |
| 6.0 | 20.0 | 21.0 | 22.1 | 8.0 | 0.1 | 22.8 |

The granular powder was filled into capsules, 200 mg per capsule. Usage can be one capsule each time, three times a day. It can cure diseases of enuresis for babies; cure frequent micturition, acute micturition, incontinence of urine for the elders; cure mental retardation and deficiency, and frequently occurring memory-barrier by embolism.

It can also be used in the form of powder, pellets, tablet, etc. It can be made as functional foods as well.

### EXAMPLE 2

Take 2000 g shell of X. sorbifolia, grind it down. Add ethyl alcohol 3 times (in volume) as much as the ground shell. Then extract materials 4 times by using Soxhlet method. Collect impregnation liquid, retrieve solvent. After decolorizing, drying and grinding, 106 g yellow-white granular powder was obtained, which contained (wt %):

| Crude | Crude | | Bunkankasaponin | | | |
|---|---|---|---|---|---|---|
| Fats | protein | Sugar | A. B. C. D. | Water | Sterols | Others |
| 6.1 | 20.2 | 22.0 | 22.6 | 8.1 | 0.1 | 20.9 |

The applications are same as those in example 1.

### EXAMPLE 3

Take 2000 g shell of X. sorbifolia, grind it down. Add methyl alcohol 3 times (in volume) as much as the ground shell. Then extract materials 4 times by using Soxhlet. Collect impregnation liquid, retrieve solvent,. After drying and grinding, 129 g brown granular powder was obtained, which contained (wt %):

| Crude | Crude | | Bunkankasaponin | | | |
|---|---|---|---|---|---|---|
| Fats | protein | Sugar | A. B. C. D. | Water | Sterols | Others |
| 6.2 | 18.8 | 22.1 | 15.2 | 9.0 | 0.1 | 28.6 |

The applications are same as those in example 1.

### EXAMPLE 4

Take 2000 g shell of X. sorbifolia, grind it. Add ethyl alcohol 3 times (in volume) as much as the ground shell. Then extract materials 4 times by using Soxhlet method. Collect impregnation liquid, retrieve solvent. The above process was carried out once again in which ethyl alcohol was substituted with n-propanol. After decolorizing, drying and grinding, 40.2 g yellow-white granular powder was obtained, which contained (wt %):

| Crude | Crude | | Bunkankasaponin | | | |
|---|---|---|---|---|---|---|
| Fats | protein | Sugar | A. B. C. D. | Water | Sterols | Others |
| 6.1 | 6.3 | 4.4 | 60.1 | 7.2 | 0.2 | 16.9 |

The applications can be same as those in example 1. Or used in multiple ingredients drugs.

### EXAMPLE 5

Take 40 g yellow-white granular powder obtained in the example, after passing them through column chromatography first and then through HPLC(high-pressure liquid chromatography) preparation, 4.0 g white crystal bodies were obtained. These crystal bodies are pure products as standard product, which contained 97% Bunkankasaponin A. B. C. D.

### EXAMPLE 6

Take 2000 g shell of X. sorbifolia, grind it down. Add ethyl alcohol 3 times (in volume) as much as the ground shell. Then extract materials 4 times by using Soxhlet method. Collect impregnation liquid, retrieve solvent. Add ethyl acetate to the liquid. Extract the liquid 4 times. Remove solvent and retrieve ethyl acetate. After drying and grinding, 28 g brown granular powder was obtained, which contained (wt %):

| Crude | Crude | | | | |
|---|---|---|---|---|---|
| Fats | protein | Sugar | Water | Sterols | Others |
| 6.2 | 21.3 | 22.6 | 10.1 | 2.8 | 37.0 |

This powder can be made as drugs in the form of powder, pellets, tablet, etc. to cure diseases of glycosuria and cancer, or as functional foods to increase immunity ability of resistance to activity of glycosuria and tumor.

### EXAMPLE 7

Take the 28 g powder obtained in the example 6, through column chromatography and crystallization, 200 mg white crystal bodies was obtained. The crystal bodies are refined product of the two kinds of sterol (dry product contains 99% of sterol) and can be as standard product.

1. Extracting materials from the shell of Xanthoceras sorbifolia Bunge and applying the extracted materials to make drugs for curing mental retardation and deficiency, dementia, enuresis, increasing body's ability of resistance to activity of glycosuria, and to make functional foods.

2. The extracted materials from the shell of Xanthoceras sorbifolia Bunge to make drugs and functional foods for enhancing brain functions. According to claim 1, the extracted materials include 2˜10 percent of crude fats, 15˜20 percent of crude proteins, 15˜25 percent of Bunkankasaponin A. B. C. D., 20˜30 percent of sugar and 7˜12 percent of water.

3. The extracted materials from the shell of Xanthoceras sorbifolia Bunge to make drugs and functional foods for enhancing brain functions, according to claim 1. Also, the extracted materials include two kinds of sterols: (3β,5α,20R,24S)-stigmasta-7, trans-22-dien-3-ol and (3β,5α,20R,24R)-stigmasta-7-en-3-ol.
